(19) United States
(12) Patent Application Publication
(10) Pub. No.: US 2022/0133191 A1
(43) Pub. Date: May 5, 2022

(54) OPTICAL METHODS TO MODULATE LOCAL BLOOD PERFUSION AT THE SKIN

(71) Applicant: Verily Life Sciences LLC, South San Francisco, CA (US)

(72) Inventors: Habibullah Ahmad, Sunnyvale, CA (US); Timothy Stowe, Alameda, CA (US); Michael Chen, Sunnyvale, CA (US)

(73) Assignee: Verily Life Sciences LLC, South San Francisco, CA (US)

(21) Appl. No.: 17/453,461

(22) Filed: Nov. 3, 2021

Related U.S. Application Data
(60) Provisional application No. 63/109,490, filed on Nov. 4, 2020.

(51) Int. Cl. A61B 5/15; A61B 5/151; A61B 5/00

(52) U.S. Cl. CPC A61B 5/150076; A61B 5/150022; A61B 5/742; A61B 5/7405; A61B 5/151

(57) ABSTRACT

Methods and systems for collecting blood samples are described. The disclosed methods and systems employ exposure of the skin surface at a sampling location to electromagnetic radiation, such as blue light, to induce vasodilation in the skin in order to increase a rate of capillary perfusion and blood collection. Following or during the exposure process, the skin at the sampling location can be pricked with one or more lancets to generate capillary perfusion sites for the blood collection process. Following collection of a blood sample, some of the disclosed devices and methods can optionally use heat or infrared electromagnetic radiation to increase a clotting rate to close the capillary perfusion sites.

### CROSS-REFERENCE TO RELATED APPLICATIONS

This application claims the benefit of and priority to U.S. Provisional Application No. 63/109,490, filed on Nov. 4, 2020, which is hereby incorporated by reference in its entirety.

### TECHNICAL FIELD

The present disclosure generally relates to obtaining capillary blood, and more specifically, but not necessarily limited to, devices and methods that utilize lancets to facilitate capillary blood extraction and collection using optical methods to modulate perfusion.

### BACKGROUND

Collection of capillary blood is useful in a variety of circumstances. For example, relatively small amounts of blood obtainable through capillary blood collection can be used to test blood sugar levels or other characteristics that are useful for diagnosing conditions relevant to a patient and informing an appropriate course of treatment.

Typically, to collect capillary blood, the skin is pricked (e.g., with a lancet) to inflict a small wound. This allows a small amount of blood to exit through the wound from capillary blood vessels near the surface of the skin. The small size of the wound allows the healing process to begin quickly, such that the blood flow usually stops after a short interval. However, the wound site may be tender afterwards, leading to discomfort, especially if a large size of lancet is utilized in an effort to obtain a sufficiently large blood sample for conducting a particular analysis.

### SUMMARY

Described herein are methods and systems for collecting blood samples, such as capillary blood from near to the skin surface. Collection of capillary blood may be a slow process in some cases, so techniques for increasing a rate of capillary perfusion may be desirable. The disclosed methods and systems employ exposure of the skin surface at a sampling location to electromagnetic radiation to induce vasodilation in the skin in order to increase a rate of capillary perfusion and blood collection.

Exposing cells and circulating biomolecules to electromagnetic radiation can stimulate nitric oxide release. When blood vessels are exposed to nitric oxide, the blood vessels can relax, increasing the cross-sectional diameter through which blood can flow (vasodilation). Thus, by exposing the skin of a sampling location to electromagnetic radiation for a period of time prior to blood collection, vasodilation can be induced at the sampling location, increasing a rate at which capillary perfusion occurs from wounds in the skin surface, and shortening a duration of a blood collection process.

Shorter wavelength electromagnetic radiation, such as ultraviolet light or blue light, is typically more effective at releasing nitric oxide and causing vasodilation than longer wavelength electromagnetic radiation, but shorter wavelength electromagnetic radiation can cause side-effects, such as sunburn and DNA damage, which may be undesirable. In general, various wavelengths of visible light can be useful for inducing vasodilation, but blue light may be most effective.

Electromagnetic radiation, such as blue light, can be generated using a variety of light sources. Light emitting diodes are one type of light source contemplated herein, as light emitting diodes generating blue light are readily available and easily incorporable in electronic devices. A variety of wavelength of light emitting diodes are available, such as, but not limited to, those generating light having a wavelength or central wavelength of 365 nm, 370 nm, 385 nm, 390 nm, 395 nm, 400 nm, 405 nm, 410 nm, 428 nm, 430 nm, 445 nm, 450 nm, 455 nm, 457 nm, 460 nm, 461 nm, 462.5 nm, 464 nm, 465 nm, 466 nm, 468 nm, 469 nm, 470 nm, 471 nm, 472 nm, 475 nm, 476 nm, 476.5 nm, 480 nm, 485 nm, or 505 nm.

Devices and methods disclosed herein use lancets to generate one or more wounds or capillary perfusion sites after light exposure to allow for collection of a blood sample. Following collection of a blood sample, some of the disclosed devices and methods can optionally use heat or infrared electromagnetic radiation to increase a clotting rate to close the capillary perfusion sites following the collection of blood.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 shows an example of a device for obtaining blood from a subject, according to some examples of the present disclosure.

FIG. 2 shows an example of a light emission unit, according to some examples of the present disclosure.

FIG. 3 shows an example of a blood collection device including a light emission unit and a blood collection unit, according to some examples of the present disclosure.

FIG. 4 shows an example of another blood collection device including integrated light emission and blood collection units, according to some examples of the present disclosure.

FIG. 5 shows an example of another blood collection device including integrated light emission and blood collection units, according to some examples of the present disclosure.

FIG. 6 illustrates an example of the device shown in FIG. 5 in a state during actuation of the lancets, according to some examples of the present disclosure.

FIG. 7 illustrates an example of the device shown in FIG. 5 in a state after actuation of the lancets and during blood collection, according to some examples of the present disclosure.

FIG. 8 illustrates an example of the device shown in FIG. 5 in a state after completion of collection of a blood sample and during activation of a heater, according to some examples of the present disclosure.

FIG. 9 shows a flow chart that illustrates an example of a process for collection of a blood sample, according to some examples of the present disclosure.

### DETAILED DESCRIPTION

Examples are described herein in the context of systems for extracting and collecting blood. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

In an illustrative example, a subject, such as a person with a medical condition, may need to provide a certain quantity of blood, for example to facilitate a diagnosis. Some blood collection procedures may be inconvenient, painful, or associated with various undesirable aspects. For example, collection of blood by a phlebotomist may require the subject to travel to a laboratory or other facility, where the subject can anticipate a hypodermic needle being inserted into a vein and the blood sample collected. Some individuals may have an adverse reaction to the sight of a needle or blood and may experience pain associated with the venipuncture.

Another blood collection method, typically used for collection of smaller amounts of blood, may use a fingerstick, where a lancet is used to pierce and wound the skin of a subject's finger for collection of blood from capillaries in the finger via the wound. Since there are many nerve endings in fingertips, piercing the skin with a lancet can cause considerable pain. In some cases, the amount of blood collected may be insufficient and so multiple fingersticks may be needed, potentially increasing overall pain.

Capillary blood collection may be performed at other locations on the body of a subject. For example, lancing the skin on the forearm or upper arm may be associated with significantly less pain than a fingerstick since there are generally fewer nerve endings in the upper arm than in the finger. In some cases, the amount and size of capillaries on the arm may be less than in the fingers, so it may be more difficult, time intensive, or require more wounds to collect the same amount of blood from the arm than from a finger.

One technique for increasing the amount of blood that can be collected from capillaries in the skin is to induce vasodilation. Vasodilation can be induced using a number of techniques, such as application of chemical vasodilation agents, for example ethanol and capsaicin, or application of heat. Heat application, while useful for causing vasodilation, can also increase the rate of clotting, which can counteract the vasodilation effects when used for collection of capillary blood.

Nitric oxide (NO), is another chemical compound that is capable of causing vasodilation. Nitric oxide is a naturally occurring signaling molecule with a short half-life in the blood (e.g., a few seconds) and is produced by a variety of different cells in the body. In some cases, nitric oxide containing or nitric oxide releasing topical agents can be applied to the skin of a subject to induce vasodilation, but the presence of the topical agents can contaminate a blood sample collected from capillary perfusion sites. Nitric oxide can be released into the blood by exposure of cells to ultraviolet and visible electromagnetic radiation, with shorter wavelengths (e.g., ultraviolet or blue light) inducing a stronger release than longer wavelengths (e.g., red light). By exposing the skin of a subject with ultraviolet or visible electromagnetic radiation prior to and/or during collection of blood from a capillary perfusion site, vasodilation can be induced, increasing the rate at which capillary blood can be collected.

When a blood sample is to be collected from a subject, the subject may use devices or methods according to this disclosure to obtain the appropriate amount of blood. In some examples, a device for collecting capillary blood can be attached to the subject's skin, and optionally with creation of a seal. The device can flood the skin of a subject with electromagnetic radiation (e.g., ultraviolet or visible light) for a period of time to induce NO-mediated capillary vasodilation. The period of time may be short, such as a few seconds, or longer, such as a few minutes or tens of minutes, to ensure a desirable amount of vasodilation occurs. The period of time may generally be the period needed for exposing the skin to electromagnetic radiation in an amount sufficient to induce NO-mediated vasodilation. After a desired exposure time, the skin surface of the subject at the sampling location can be lanced by activating one or more lancets to prick the skin and generate one or more capillary perfusion sites to start blood flow for collection by the device. In some cases, a light emission unit used to expose the skin with electromagnetic radiation may be integrated in a device for collecting capillary blood with a blood collection unit or the light emission unit may be a separate component from the blood collection unit.

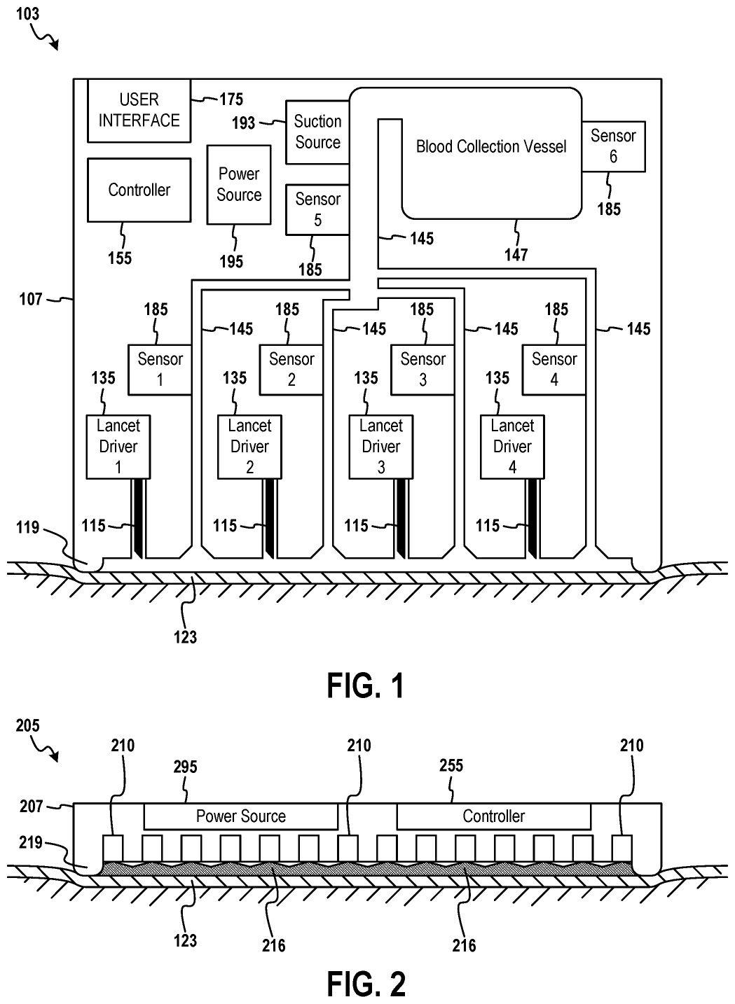

FIG. 1 shows an example device 103 for obtaining blood from a subject (i.e., a blood collection unit). The device 103 is shown with a housing 107, which may include respective components of the device 103. Although the components in FIG. 1 are all shown within the housing 107, in some examples, at least some components may be positioned at least partially outside of the housing 107. Moreover in some examples, some components may be removable or replaceable, e.g., formed as cartridges or other consumable parts that may be installed prior to use and/or replaced upon use.

The device 103 includes a plurality of lancets 115. In FIG. 1, four lancets 115 are shown, although any suitable multiple number of lancets 115 may be included, such as but not limited to one, two, three, four, or more than four. The lancets 115 may be any suitable size or form factor for penetrating or wounding the skin to generate capillary perfusion sites for collection of blood by the device 103. For example, the lancets may correspond to needles with round cross-sections (e.g., such as may be common in 28 gauge or 27 gauge or other fine gauge needles on up through larger gauges, such as 20 gauge), optionally with a beveled tip, or structures with a form factor like a blade (e.g., such as may be common in larger gauge lancets of 18 gauge, 17, gauge, or larger). Moreover, although the lancets 115 in FIG. 1 are all depicted as approximately the same size, a variety of sizes or form factors can be utilized in conjunction with one another.

The lancets 115 may be individually addressable. For example, the lancets 115 may be individually drivable, which may minimize a number of wounds that may be inflicted for obtaining a suitable quantity of blood during a blood draw procedure performed by the device 103.

The housing 107 shown in FIG. 1 includes one or more projections 119 that may form a ring or other appropriate structure to form a seal on a subject's skin 123 during the blood collection process. For example, the projections 119 may correspond to ridges or other structures that may generate a sealed volume and/or area between the skin 123 of the subject and the housing 107. The sealed volume or area may surround a volume in which blood released by wounds or capillary perfusion sites, imparted by operation of the lancets 115, may be collected and directed to a suitable location within the device housing 107 or associated structure. Projections 119 are optionally separable from housing 107 for removal of device 103 to allow projections 119 to remain attached to skin of the subject and replacement of device 103 with another device or to allow attachment of device 103 to projections from another device or component.

The device 103 may include one or more lancet drivers 135. The lancet drivers 135 may include any suitable parts or components that can cause extension and/or retraction of the lancets 115 to produce a respective blood extraction wound or capillary perfusion site. Suitable examples may include chemical charges (such as propellants activatable to generate pressure to push a piston or otherwise impart force for driving), springs, motors, or other force-imparting mechanisms.

In FIG. 1, the device 103 is shown with four lancet drivers 135, e.g., each respectively coupled with a respective lancet 115. Although the lancet drivers 135 are shown in a one-to-one relationship with the respective lancets 115, other arrangements are possible. In some examples, a single lancet driver 135 may be capable of moving and/or cycling between different lancets 115 for respective actuations of the lancets 115, such as by relative motion between the lancets 115 and the lancet driver 135.

A flow conduit network is also shown in FIG. 1 for allowing blood from lancet-imparted wounds or capillary perfusion sites to flow or otherwise be collected into the device 103. For example, the flow conduit network can include any suitable number of conduits 145 or flow paths that may direct blood from a wound or capillary perfusion site and into a blood collection vessel 147. The flow conduit network may be part of, connected with, or extend into the housing 107. In some examples, the flow conduit network may include portions or components sized for imparting particular microfluidic flow properties such as wicking, capillary action, or other flow properties or dynamics that may facilitate movement of blood through the device 103. The flow conduit network may include any suitable structure or architecture, such as with conduits 145 individually adjacent to each lancet or with conduits 145 between lancets, or the like. In some examples, such as shown in FIG. 1, various conduits 145 may combine or otherwise channel blood received from individual conduits 145 into an aggregate flow conduit, although in some examples, individual conduits may individually feed into the blood collection vessel 147 without combining first through an aggregate conduit. Moreover, although FIG. 1 depicts a single blood collection vessel 147, more than one may be included, e.g., optionally each corresponding to different lancets 115 or combinations thereof.

The blood collection vessel 147 can include a storage cavity in fluid communication (e.g., via the flow conduit network) with the housing 107 (e.g., to a portion adjacent the sealed volume formed by engagement of the subject's skin 123 with the projection 119 or other part of the housing 107). The blood collection vessel 147 may have any suitable volume. Useful example volumes may range from 20 μl to 5 ml, such as from 50 μl to 2 ml. The blood collection vessel 147 may receive blood extracted from the subject through the skin 123 of the subject. The blood collection vessel 147 is shown in FIG. 1 schematically within the housing 107. However, the blood collection vessel 147 may be at least partially out of the housing, e.g., extending at least partially out or coupled via a hose or tube that extends out of the device housing 107. In some examples, the blood collection vessel 147 may be removable and/or replaceable with a different blood collection vessel 147. For example, in use, the blood collection vessel 147 may correspond to a vial or other structure that may be removed upon filling to a suitable amount. In some examples, the blood collection vessel 147 may be coupled with or include appropriate gaskets or other sealing structures, for example, to facilitate removal of the blood collection vessel 147 without leaking collected blood.

The device 103 depicted in FIG. 1 also includes a controller 155. The controller 155 may include appropriate components for controlling lancet drivers 135 or other parts of the device 103. For example, the controller 155 may be implemented such that each lancet 115 is individually addressable. The controller 155 may be in data communication with other components, such as via wired or wireless communication pathways. In some examples, the controller 155 may be capable of performing different functions including, but not limited to, keeping time, receiving inputs, making comparisons and/or determinations, and/or providing command signals to trigger operations or other acts of other components within the device 103.

The controller 155 may correspond to any suitable structure for enabling associated functions. In some examples, the controller 155 may correspond to an application-specific integrated circuit (“ASIC”) defined on a field-programmable gate array (“FPGA”) or other form of processor.

In FIG. 1, a user interface 175 is also shown. In use, the user interface 175 may provide input to the controller 155. For example, the user interface 175 may be or include a touch screen or button or other input mechanism. In some examples, the user interface 175 may allow a user (e.g., the subject or a clinician working with the subject) to input a command or otherwise control the device 103 to initiate operation of the device 103. In some examples, the user interface 175 may be in communication with, such as via wireless communication, or comprise an application on a computer or a mobile device (e.g., a smartphone or tablet computer) for providing input to the device 103.

The device 103 may further include one or more sensors 185. In general, the sensors 185 may provide information for monitoring activity of the device 103 which may be used to facilitate determinations about subsequent actions of the device 103.

In some examples, the sensors 185 may correspond to volumetric sensors, for example, which may provide information about a rate and/or amount of blood received by the device 103. Suitable examples may include, but are not limited to sensors that operate on optical, thermal, and/or conductive principles. As one illustrative example, an optical sensor at a particular location may detect a change in color or other light characteristic when blood reaches that location and so indicate that an amount of blood sufficient to reach that location has been obtained. Multiple sensors 185 may be placed at different positions along a route to allow a variety of amounts to be detected.

The sensors 185 utilized may be any combination of different kinds of volumetric sensors. In some examples, different types of volumetric sensors may be utilized for redundancy purposes. Additionally, any suitable arrangement of the sensors 185 may be utilized. For example, although in FIG. 1 a respective sensor 185 (Sensors 1-4) is shown on or adjacent to a respective conduit 145, the device 103 may additionally or alternatively include an aggregate sensor 185, such as Sensor 5, that may provide information about an aggregated amount of blood. Further, a sensor 185 may be positioned in a position to monitor the volume of blood in blood collection vessel 147, such as Sensor 6 in FIG. 1. In some cases, device 103 may be configured to generate an audible or visible alert or other indicator of completion of collecting the blood sample, such as when Sensor 6 indicates a target amount of blood has been collected.

The device 103 may include one or more other components to facilitate functions or operations of the device 103. For example, in FIG. 1, the device 103 is shown with a suction source 193. The suction source 193 may facilitate flow of received blood toward the blood collection vessel 147. The suction source 193 may correspond to a pump or a vacuum container capable of drawing a vacuum for moving or imparting flow in a particular direction of blood through the device 103. Other example components may include a power source 195, which may comprise a battery, supercapacitor, or wireless power coil, for example, to provide power to one or more components, such as lancet drivers 135, controller 155, user interface 175, sensors 185, suction source 193.

In some examples, elements in the housing 107 may be remotely activated. For example, one or more components associated with the device 103 may receive a wireless command from a remote device, e.g., which may be any suitable device with a wireless transmitter, such as a smartphone, smartwatch, blood pressure sensor, continuous glucose monitor (“CGM”), etc. Such remote devices may be handheld or wearable devices or larger devices, such as one or more sensing systems as may be found in a hospital or other medical office or clinic. Suitable wireless communication mechanisms include Bluetooth®, Bluetooth® low-energy (“BLE”), WiFi, near-field communications (“NFC”), etc.

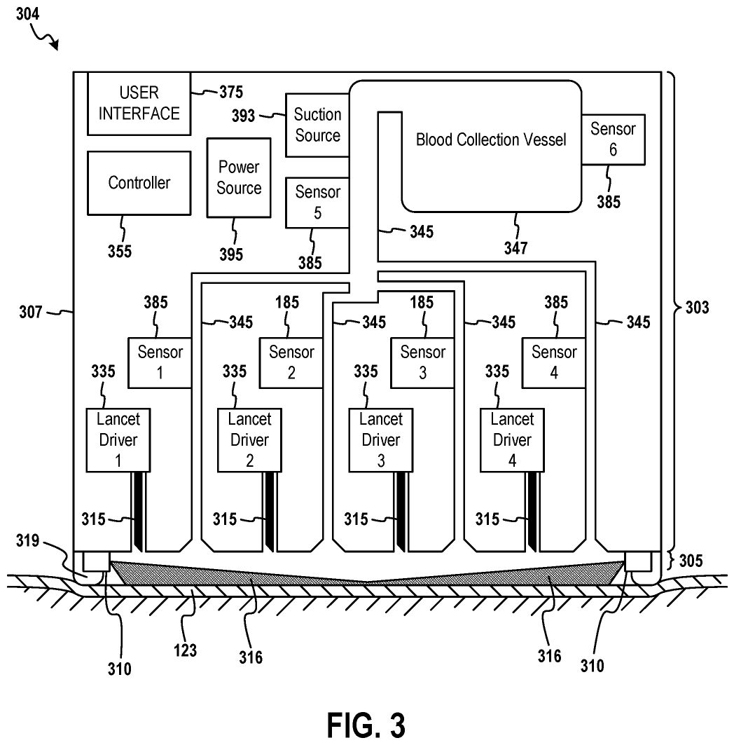

Device 103 may contain other components than those depicted in FIG. 1. In general, the components of device 103 depicted in FIG. 1, or device 103 itself, may be referred to as a blood collection unit. In some cases, device 103 may contain a light emission unit for subjecting the skin 123 of the subject to electromagnetic radiation to induce vasodilation, as described above, and also in further detail below with respect to FIGS. 3-5. In some cases, a light emission unit for subjecting the skin 123 of the subject to electromagnetic radiation to induce vasodilation may be separate from the blood collection unit.

FIG. 2 shows an example device comprising a standalone light emission unit 205. Light emission unit 205 is shown with a housing 207, which may include respective components of light emission unit 205. Although the components in FIG. 2 are all shown within the housing 207, in some examples, at least some components may be positioned at least partially outside of the housing 207. Moreover in some examples, some components may be removable or replaceable. The housing 207 include one or more projections 219 that may form a ring or other appropriate structure to form a seal on a subject's skin 123 during the light exposure process. Projections 219 are optionally separable from housing 207 for removal of light emission unit 205 to allow projections 219 to remain attached to skin of the subject and replacement of light emission unit 205 with another device (e.g., a blood collection device).

The light emission unit 205 includes a plurality of light sources 210. In FIG. 2, fourteen light sources 210 are shown, although any suitable number of individual light sources 210 may be included, such as any integer from 1 to 100 or more. Light sources 210 may be light emitting diodes or other light sources, producing electromagnetic radiation 216 directed toward skin 123. As noted above, the electromagnetic radiation 216 may induce vasodilation within skin 123. Various wavelength of electromagnetic radiation 216 can cause release of nitric oxide and concomitant vasodilation, but wavelengths of electromagnetic radiation in the ultraviolet and visible regions, such as from about 360 nm to 520 nm, may be most effective for inducing vasodilation. In particular ultraviolet light may be strongly associated with nitric oxide release, but other deleterious effects may be associated with ultraviolet light, so longer wavelengths may be preferred for avoiding the deleterious effects. For example, blue light, such as electromagnetic radiation having wavelengths of from about 400 nm to 450 nm or about 420 nm to about 450 nm may be used for release of nitric oxide and/or vasodilation. In some cases, other wavelengths of electromagnetic radiation, such as other colors of visible light, such as green light or red light or white light, may be useful for releasing nitric oxide in the blood and/or causing vasodilation, but blue light may have the strongest effect in the visible region.

A variety of ultraviolet, visible, blue, or green light emitting diodes are commercially available, such as those used for generating electromagnetic radiation having a wavelength or a central wavelength of 365 nm, 370 nm, 385 nm, 390 nm, 395 nm, 400 nm, 405 nm, 410 nm, 428 nm, 430 nm, 445 nm, 450 nm, 455 nm, 457 nm, 460 nm, 461 nm, 462.5 nm, 464 nm, 465 nm, 466 nm, 468 nm, 469 nm, 470 nm, 471 nm, 472 nm, 475 nm, 476 nm, 476.5 nm, 480 nm, 485 nm, or 505 nm. Other wavelengths or wavelength ranges for light emitting diodes may be used.

In some cases, exposure of the skin 123 to electromagnetic radiation 216 may result in an increase in temperature of the skin. It may be desirable to keep a temperature change of the skin to a minimum during exposure of the skin 123 to electromagnetic radiation 216, so as to keep a clotting rate of blood in the skin 123 to a minimum to support collection of a blood sample. In some cases, exposure of the skin 123 to electromagnetic radiation 216 may raise a temperature of the skin 123 by less than 5° C., such as less than 3° C., less than 2° C., less than 1° C. or 0° C. Optionally, a temperature sensor is included in light emission unit 205 and coupled to controller 255 to determine if a temperature change is induced in skin 123 by electromagnetic radiation 216 and to terminate light emission, at least temporarily, to limit the overall temperature increase in skin 123.

In some cases, exposure of the skin 123 to electromagnetic radiation 216 may be paired with topical treatments on the skin 123 as a way to cause additional release of nitric oxide to the blood. For example, nitric oxide precursors, such as NaNO₂ may be applied to the skin and subjected to electromagnetic radiation 216 to cause the nitric oxide precursors to release nitric oxide. In this way, two pathways for release of nitric oxide into the blood can be mediated by electromagnetic radiation 216. However, as noted above, the application of topical treatments on the skin 123 may result in contamination of collected blood by components of the topical treatment, in some cases, but exposure of the skin 123 to electromagnetic radiation 216 alone may avoid contaminating the blood in this way.

Light emission unit 205 is further illustrated in FIG. 2 as including a controller 255 and a power source 295. The controller 255 may include appropriate components for controlling light sources 210 or other parts of the light emission unit 205. For example, the controller 255 may be implemented such that each light source 210 is individually addressable. The controller 255 may be in communication with other components, such as via wired or wireless communication pathways. In some examples, the controller 255 may be capable of performing different functions including, but not limited to, keeping time, receiving inputs, making comparisons and/or determinations, and/or providing command signals to trigger operations or other acts of other components within the light emission unit 205. In some examples, the controller 255 may be programmed to control the light emission by light sources 210, such as a duration or intensity of electromagnetic radiation 216. Example ranges of duration of light emission by the light source 210 may be from 5 seconds to 30 minutes, or various values between these. In some cases, short durations of light emission may be sufficient for inducing vasodilation, such as from 5 seconds to 30 seconds, for example. In some cases, longer durations of light emission may be desirable for inducing vasodilation, such as from 1 minute to 15 minutes, for example. The duration of light emission may be pre-programmed or adjustable.

In some cases, light sources 210 may be controlled such that electromagnetic radiation 216 provides a continuous flood exposure on skin 123 or such that electromagnetic radiation 216 provides a pulsed exposure on skin 123. In some cases, pulsed exposure may be useful for generating higher intensities of electromagnetic radiation 216 during a pulse event than may be achievable during a continuous flood exposure. In some cases, higher intensities of electromagnetic radiation 216 are useful for increasing a penetration depth of electromagnetic radiation 216 into skin 123, which can result in deeper or more efficient vasodilation for subsequent collection of blood.

The controller 255 may correspond to any suitable structure for enabling associated functions. In some examples, the controller 255 may correspond to an application-specific integrated circuit (ASIC) defined on a field-programmable gate array (“FPGA”) or other form of processor. In some examples, controller 255 may communicate, such as via wireless communication, with an external device or program, such as an application on a computer or a mobile device (e.g., a smartphone or tablet computer) for providing input or instructions to the light emission unit 205.

Light emission unit 205 may contain other components besides those shown. For example, one or more sensors, a user interface, optical elements (lenses, diffusers, reflectors), or other elements may be included in light emission unit 205.

While FIG. 1 shows a blood collection unit and FIG. 2 shows a light emission unit as separate devices, these components may be combined into a single device. FIG. 3 shows a device 304 combining a blood collection unit 303 and a light emission unit 305. Although blood collection unit 303 and light emission unit 305 are labeled as individual regions of device 304, some components of device 304 may be shared between blood collection unit 303 and light emission unit 305. Device 304 may contain the same components as device 103, along with other components. Device 304 may contain the same components as light emission unit 205, along with other components.

In some cases, light emission unit 305 and blood collection unit 303 may be separable components, which may stack or nest with one another. For example, in some cases, light emission unit 305 may comprise a ring-shaped device that sits between blood collection unit 303 and skin 123, optionally integrating a projection 319 for engaging the skin 123 to form a seal. Such a separable configuration may be useful for allowing light emission unit 305 to be cleaned, sterilized, and/or reused. In some cases, light emission unit 305 and blood collection unit 303 may be integrated components, residing together inside housing 307. Further examples of devices integrating a light emission unit and a blood collection unit are described below with reference to FIGS. 3-5.

Similar to device 103 depicted in FIG. 1, device 304 may comprise one or more lancets 315, one or more lancet drivers 335, one or more conduits 345 for transporting blood to a blood collection vessel 347, a controller 355, a user interface 375, one or more sensors 385, a suction source 393, and a power source 395. These components may be operable and/or configured similar or identical to the configuration of corresponding components in device 103 depicted in FIG. 1.

Device 304 also comprises one or more light sources 310. Light sources 310 may be the same as or different from light sources 210 described with respect to FIG. 2. In the configuration shown, light sources 310 may be arranged in a ring configuration, such as adjacent to, within, or nearby to projections 119. Although two light sources 310 are shown in FIG. 3, additional light sources 310 may be present. Light sources 310 are depicted in FIG. 3 as generating electromagnetic radiation 316, and may be configured similarly or identical to corresponding components in light emission unit 205 depicted in FIG. 2. In some cases, the electromagnetic radiation 316 may be directed towards a location of lancets 315 or a tip of lancets 315 in an extended configuration, such as towards the skin 123.

Device 304 may also comprise one or more components in addition to those depicted in FIG. 3. For example, device 304 may comprise one or more indicators, such as an optical indicator (e.g., a light) or an audio indicator (e.g., a speaker, buzzer, or bell). Such indicators may be useful for providing indications of completion or collection of a light exposure process or a blood collection process. In some cases, such an indictor may be or comprise a component of user interface 375. Such indicators may also provide other information, such as signifying that device 304 is ready for activation, properly placed for use, that a suitable seal with skin 123 is established, or the like.

Although light sources 310 are depicted in FIG. 3 as activated and generating electromagnetic radiation 316, it will be appreciated that light sources 310 may not continually generate electromagnetic radiation 316 and can be activated and deactivated. Activation of light sources 310 may be performed by controller 355. In some cases, light sources 310 may be automatically activated upon or after placement of housing 307 on skin 123 or upon or after forming a seal between housing 307 and skin 123. In some cases, light sources 310 may be automatically deactivated upon or after removal of housing 307 from skin 123 or upon or after breaking the seal between housing 307 and skin 123. In some cases, light sources 310 may be automatically deactivated after a fixed amount of time.

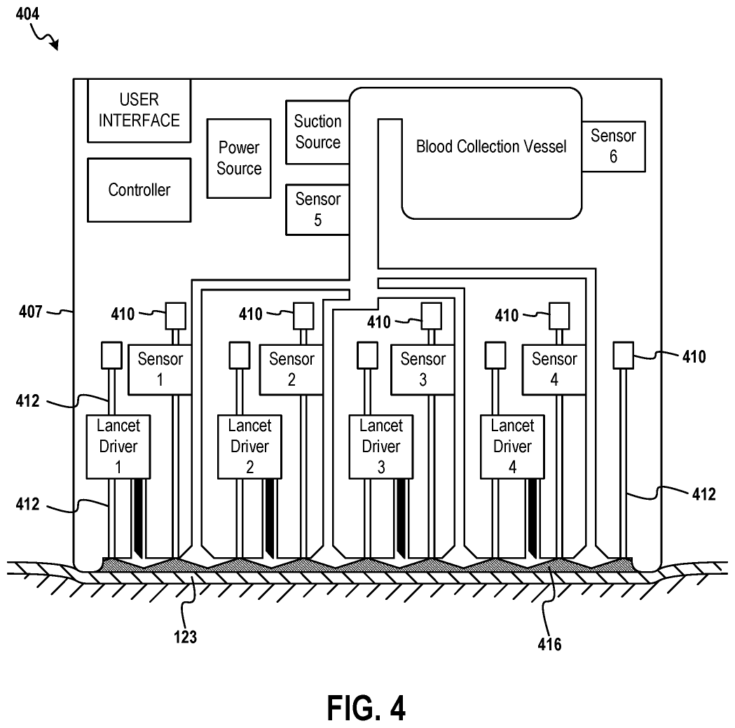

Other configurations or arrangements of a light emission unit and a blood collection unit may be used. For example, FIG. 4 shows another example device 404 where a light emission unit components and a blood collection components are integrated. Device 404 may generally include the same or identical components as device 103 shown in FIG. 1, light emission unit 205 shown in FIG. 2, or device 304 shown in FIG. 3, but with more or fewer components and/or changes in the arrangement of some components. In FIG. 4, the light sources 410 are shown internal to the housing 407 of device 404, with light conduits 412 used to transmit electromagnetic radiation 416 generated by the light sources 410 towards skin 123. Light conduits 412 may be any suitable structure that can transport electromagnetic radiation generated by light sources 410 to a volume between housing 407 and skin 123. In some examples, light conduits 412 may comprise light tubes, light pipes, optical fibers, waveguides, prisms, free space, or other optical media used for allowing transmission of electromagnetic radiation. Light conduits 412 are depicted in FIG. 4 as straight sections, but may be curved or otherwise provided in any suitable arrangement to allow emission of electromagnetic radiation 416 towards skin 123.

In FIG. 4, nine light sources 410 and nine light conduits 412 are depicted, with one-to-one correspondence, but other configurations can be used, such as where a light conduit 412 branches and provides light generated by one light source 410 to multiple exposure areas, or where multiple light sources 410 feed light into a common light conduit 412. Light conduits 412 may comprise or include optical components, such as lenses, diffusers, reflectors, or the like to allow efficient exposure of skin 123 to electromagnetic radiation 416. Some arrangements of light sources may not use or require light conduits, such as depicted in FIG. 3.

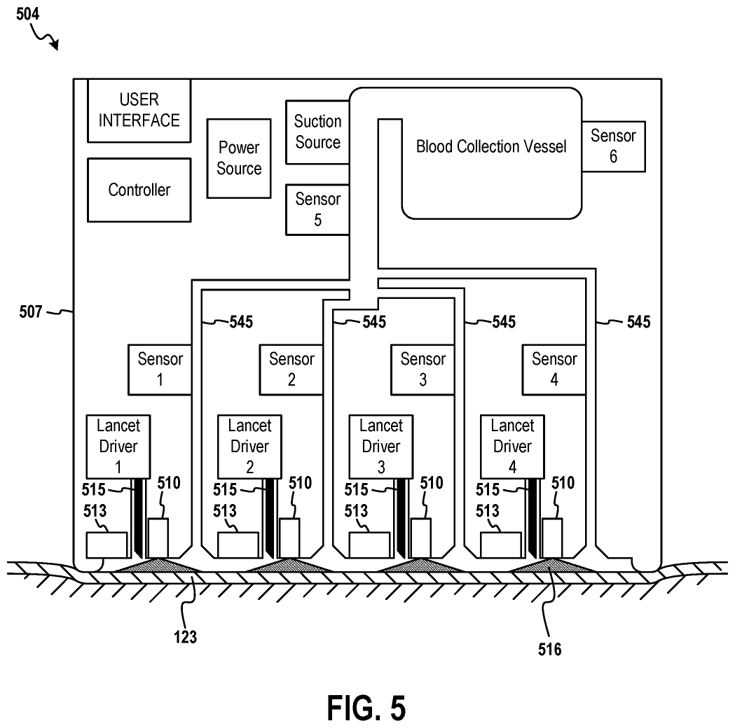

FIG. 5 shows another example device 504 where a light emission unit components and a blood collection unit components are integrated. Device 504 may generally include the same or identical components as device 103 shown in FIG. 1, light emission unit 205 shown in FIG. 2, device 304 shown in FIG. 3, or device 404 shown in FIG. 4, but with more or fewer components and/or changes in the arrangement of some components. In FIG. 5, the light sources 510 are arranged at a position on the bottom of housing 507, adjacent to lancets 515 and inlets for fluid conduits 545. In some examples, light sources 510 may comprise light emitting diodes positioned on a printed circuit board which is attached to the bottom of housing 507. Light sources 510 are depicted in FIG. 5 as generating electromagnetic radiation 516 over portions of skin 123 beneath device 504, such as portions of skin 123 where lancets 515 can create perfusion sites upon being deployed from a retracted position to an extended position.

Device 504 also includes heat sources 513, which may comprise infrared emission sources, heaters, or the like, for increasing a temperature of skin 123, such as to induce clotting or increase a rate of clotting or coagulation of blood at capillary perfusion sites at the end of a blood collection process.

Although device 504 shows four light sources 510 and four heat sources 513, any suitable number of these components may be used, for example as few as one light source 510 or as few as zero or one heat source 513, or up to 20 or 50 or 100 or more of each, may be used.

For simplicity in the description, the operation of the device 504 will be further described, but it will be appreciated that the device 103 depicted in FIG. 1, the device 304 depicted in FIG. 3, the device 404 depicted in FIG. 4, and the device 504 depicted in FIG. 5 may operate identically, or they may operate differently in some cases. Operation of device 504 may control the number of lancets 515 deployed and utilized to collect an appropriate amount of blood. Device 504 may control the number of lancets 515 based on blood flow obtained by the device, such as where only one lancet 515 is deployed at a time unless additional blood flow is needed to collect a desired amount of blood. In some cases, however, device 504 may control or deploy all lancets 515 at the same time to obtain the desired amount of blood in as short a time as possible. Further, activation of light sources 510 to expose skin 123 to electromagnetic radiation 516 may be used to induce vasodilation in skin 123 to increase a rate of perfusion from wounds generated by lancets to reduce a blood collection time.

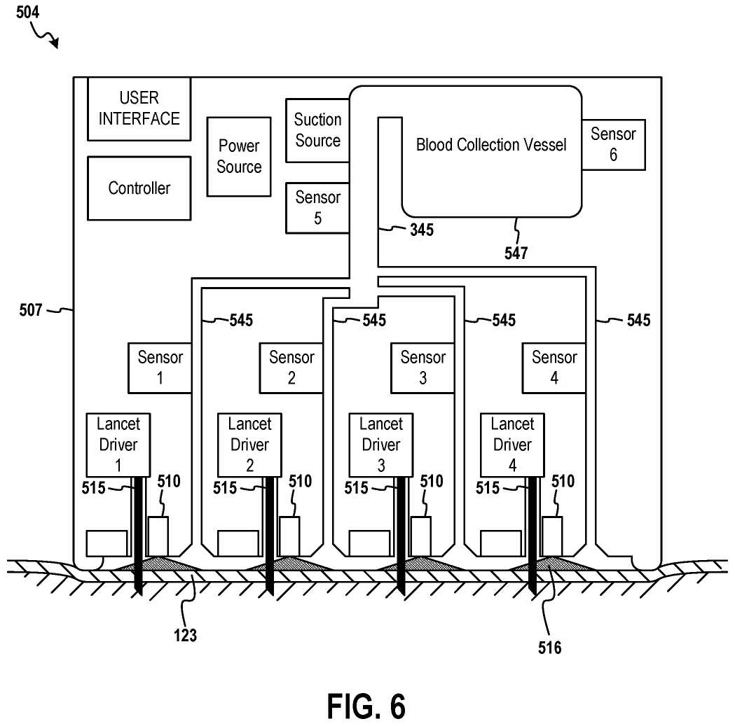

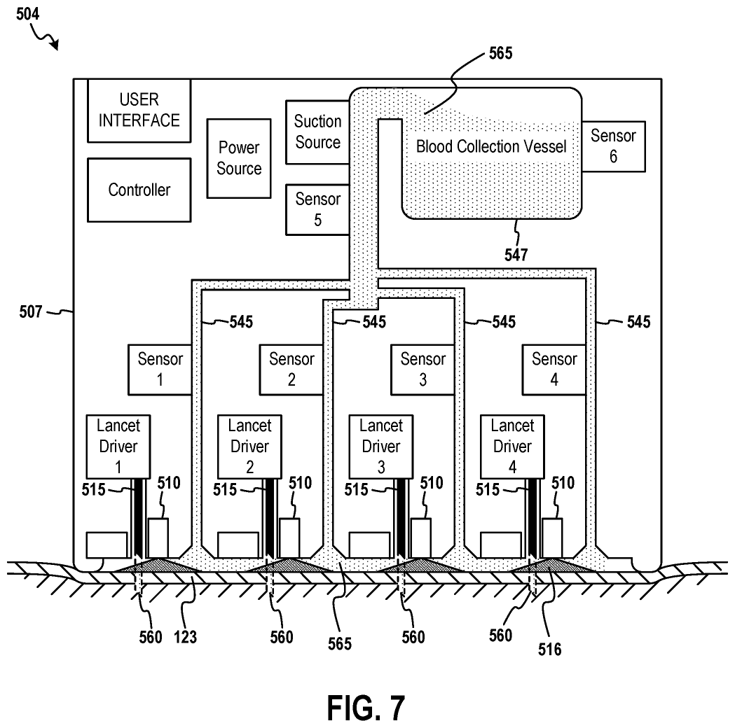

An example progression of states of the device 504 are shown in FIGS. 5-8. FIG. 5 shows an example of the device 504 in a state before the lancets 515 are actuated and while light sources 510 expose skin 123 to electromagnetic radiation 516. FIG. 6 shows an example of the device 504 in a state while the lancets 515 are actuated (e.g., in an extended position) to generate wounds (capillary perfusion sites). FIG. 7 shows an example of the device 504 in a state after actuation of lancets 515, where lancets 515 are returned to a retracted position, showing wounds 560, filling of at least some conduits 545 with blood, and collection of a blood sample in blood collection vessel 547. Although FIG. 6 and FIG. 7 show light sources 510 exposing skin 123 to electromagnetic radiation 516, some configurations may only expose skin 123 to electromagnetic radiation 516 prior to actuation of lancets 515. As depicted in FIG. 6 and FIG. 7, light sources 510 may expose skin 123 to electromagnetic radiation 516 during lancing of the skin 123 and collection of blood 565.

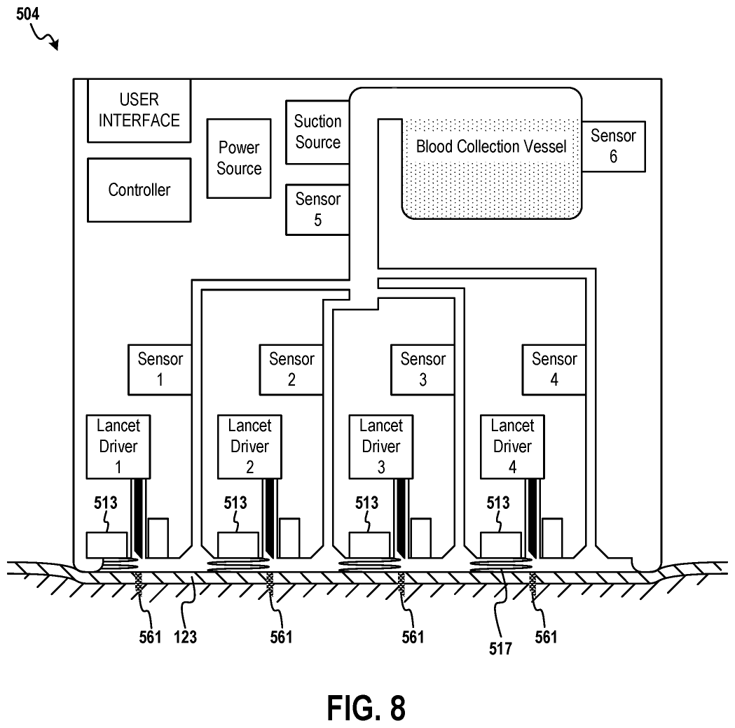

Generally, after the sample of blood 565 is collected in blood collection vessel 547, light sources 510 may stop generating electromagnetic radiation, as depicted in FIG. 8. In some examples, such as where device 504 comprises heat sources 513, skin 123 may be heated or exposed to infrared electromagnetic radiation 517 generated by heat sources 513, which can increase a clotting rate and formation of clots 561.

The devices described above may be included in a kit, such as a blood collection kit. In some examples, a kit may comprise a device or devices described herein and instructions for use. For example, a kit may comprise a device including a blood collection unit with instructions for applying the blood collection unit to a skin surface and for activating the device, such as to activate a light source to emit electromagnetic radiation to the skin surface, or to activate a lancet to generate capillary perfusion sites in the skin surface, or to activate components for collection of blood. Such a kit may optionally comprise other equipment to facilitate safe collection of blood, such as a disinfectant wipe or a bandage. Depending on the components of the device, the kit may optionally comprise a blood collection container, though the blood collection container may be incorporated into the device in some cases. Optionally, a kit may comprise an application or computer program or instructions for obtaining an application or computer program for operating or communicating with a device for the collection of blood. Other equipment may optionally be included, such as a centrifuge or a hardware device for sample monitoring during collection or shipping. Optionally, a collected blood sample may be shipped to a laboratory for analysis, and so a kit may comprise shipping materials, a shipping label, or shipping instructions.

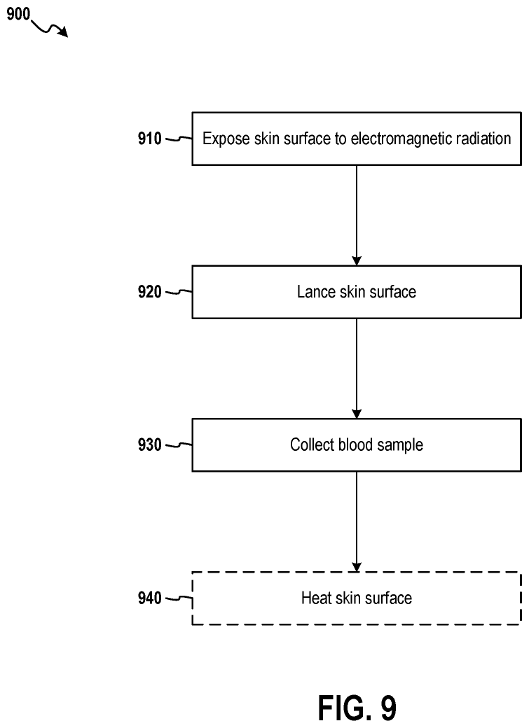

FIG. 9 shows a flow chart that illustrates a process 900 that may be performed by devices and systems described herein. At block 910, the process 900 may include exposing the skin surface of a subject to electromagnetic radiation. For example, this may correspond to exposing the skin surface to electromagnetic radiation in an amount and wavelength sufficient to induce capillary vasodilation at a sampling location of the skin, such as NO-mediated capillary vasodilation. The electromagnetic radiation may be or comprise visible light or ultraviolet light, and may particularly include blue light. The light may be generated using light emitting diodes, for example. The exposure may be a pulsed exposure or series of exposure pulses or may be a continuous flood exposure. The exposure may have a duration, prior to additional steps of process 900. Optionally, the exposure may continue during additional step of process 900.

The exposure may be initiated by interaction with a user interface of the device or system, such as by receiving input from a user interface or other input device, for example a button press. In some cases, the exposure may be automatically initiated upon the device forming a seal with the skin surface.

At block 920, the process 900 may include lancing the skin surface, such as at the sampling location to generate one or more capillary perfusion sites (i.e., wounds) from which blood may be expressed or collected. In some cases, lancing of the skin surface may occur after an amount of time of the exposing at block 910, so as to allow sufficient time during the exposing for vasodilation to occur. Lancing may occur automatically, such as automatically after some period of time following the start of exposing. In some cases, lancing may occur upon receipt of input, such as via a user interface or other input device.

At block 930, the process 900 may include collecting a blood sample, such as from the one or more capillary perfusion sites. The blood collection process may be initiated immediately following lancing of the skin at block 920 or after a delay following lancing. The blood collection process may be a passive process in which the device automatically collects blood without user or device interaction. In some cases, the blood collection process may be a process that occurs by activating components within the device, such as activating a suction source, opening a valve, or the like.

At block 940, the process 900 may optionally include heating the sampling location or the capillary perfusion sites. Heating may occur by exposing the skin surface to infrared radiation or by activating a heat source, such as a resistive heater. Heating the sampling location or capillary perfusion sites may be useful for increasing a rate of clotting, so as to seal the one or more capillary perfusion sites. In some cases, block 940 may be omitted.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure. For example, more or fewer steps of the processes described herein may be performed according to the present disclosure. Moreover, other structures may perform one or more steps of the processes described herein.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” “in some cases”, “in some examples,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Some examples in this disclosure may include a processor. A computer-readable medium, such as RAM may be coupled to the processor. The processor can execute computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices, such as programmable logic controllers (PLCs), programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example, computer-readable storage media, that may store instructions that, when executed by the processor, can cause the processor to perform the steps described herein as carried out, or assisted, by a processor. Examples of computer-readable media may include, but are not limited to a memory chip, ROM, RAM, ASIC, or any other medium from which a computer processor can read or write information. The processor, and the processing described, may be in one or more structures, and may be dispersed through one or more structures. The processor may comprise code for carrying out one or more of the methods (or parts of methods) described herein.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Every formulation or combination of components described or exemplified can be used to practice the invention, unless otherwise stated. Specific names of materials are intended to be exemplary, as it is known that one of ordinary skill in the art can name the same material differently. It will be appreciated that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

U.S. Pat. No. 9,033,898.

U.S. Patent Application Publication Nos. US 2016/0038068, US 2011/0144726, US 20180021219.

PCT International Application Publication Nos. WO 2009/125338, WO 2015/123657.

European Patent No. 2 493 536.

Ieda et al., “Photomanipulation of Vasodilation with a Blue-Light-Controllable Nitric Oxide Releaser,” J. Am. Chem. Soc. 2014, 136, 7085-7091; DOI: 10.1021/ja5020053.

Krleza et al., “Capillary blood sampling: national recommendations on behalf of the Croation Society of Medical Biochemistry and Laboratory Medicine,” Biochemica Medica 2015, 25(3), 335-358; DOI: 10.11613/BM.2015.034.

What is claimed is:

1. A method for collecting capillary blood, the method comprising:
exposing a skin surface of a subject to electromagnetic radiation in an amount sufficient to induce NO-mediated capillary vasodilation at a sampling location;
lancing the skin surface of the subject at the sampling location to generate one or more capillary perfusion sites; and
collecting a blood sample from the one or more capillary perfusion sites.

2. The method of claim 1, wherein the exposing is performed by a light emission unit comprising a light source for generating the electromagnetic radiation, and wherein the lancing and the collecting are performed by a blood collection unit having a skin sealing side, a blood collection side, and at least one movable lancet having a retracted position and an extended position.

3. The method of claim 2, wherein the light emission unit and the blood collection unit are separate devices, or wherein the light emission unit is coupled to the blood collection unit in an arrangement to direct electromagnetic radiation emitted by the light source toward at least a tip of the at least one movable lancet in the extended position.

4. The method of claim 1, wherein the electromagnetic radiation includes blue light or light having a wavelength of from 360 nm to 520 nm.

5. The method of claim 1, wherein exposing the skin surface of the subject to the electromagnetic radiation heats the skin surface by no more than 5° C.

6. The method of claim 1, wherein the exposing comprises exposing the skin surface of the subject to one or more pulses of the electromagnetic radiation or to a constant flood of the electromagnetic radiation.

7. The method of claim 1, wherein exposing the skin surface of the subject to the electromagnetic radiation occurs for a duration of time prior to the lancing, wherein the duration of time is from 10 seconds to 30 minutes or from 30 seconds to 5 minutes.

8. The method of claim 1, wherein exposing the skin surface of the subject to the electromagnetic radiation occurs during the lancing, during the collecting, or during both the lancing and the collecting.

9. The method of claim 1, wherein collecting the blood sample comprises collecting from 50 μl to 5 ml of blood or from 100 μl to 2 ml of blood.

10. The method of claim 1, further comprising, after collecting the blood sample:
exposing the skin surface of the subject to infrared electromagnetic radiation in an amount sufficient to increase a rate of clotting or coagulation of blood at the one or more capillary perfusion sites; or
heating the skin surface of the subject by a temperature sufficient to increase a rate of clotting or coagulation of blood at the one or more capillary perfusion sites.

11. The method of claim 1, further comprising:
detecting collection of a target amount of blood; and
indicating completion of the collecting, wherein indicating completion of the collecting comprises one or more of:
generating an audible indicator;
generating a visual indicator; or
stopping the exposing.

12. A device for collecting capillary blood, the device comprising:
a blood collection unit having a skin sealing side, a blood collection side, and at least one movable lancet having a retracted position and an extended position; and
a light source coupled to the blood collection unit in an arrangement to direct emitted electromagnetic radiation toward a tip of the at least one movable lancet in the extended position.

13. The device of claim 12, wherein the light source is a source of light having a wavelength sufficient to induce NO-mediated capillary vasodilation at a sampling location of a skin of a subject.

14. The device of claim 12, wherein the light source is a blue light source or a source of light having a wavelength of from 360 nm to 510 nm.

15. The device of claim 12, wherein the light source is a pulsed light source or a constant flood source.

16. The device of claim 12, further comprising:
an infrared light source coupled to the blood collection unit in an arrangement to direct emitted infrared electromagnetic radiation to increase a rate of clotting or coagulation of blood; or
a heat source coupled to the blood collection unit in an arrangement to increase a temperature at the skin sealing side by an amount sufficient to increase a rate of clotting or coagulation of blood.

17. The device of claim 12, further comprising:
one or more processors; and
a non-transitory computer readable storage medium including instructions that, when executed by the one or more processors, cause the one or more processors to perform operations including:
initiating emission of electromagnetic radiation by the light source;
initiating lancing of a skin surface of a subject at a sampling location using the at least one movable lancet to generate one or more capillary perfusion sites by temporarily engaging the at least one movable lancet from the retracted position to the extended position; and
initiating collection of a blood sample from the one or more capillary perfusion sites.

18. A system for collecting capillary blood, the system comprising:
a blood collection unit having a skin sealing side, a blood collection side, and at least one movable lancet having a retracted position and an extended position; and
a light emission unit comprising a light source for directing emitted electromagnetic radiation toward a skin surface.

19. The system of claim 18, wherein the light emission unit is a physically separate component from the blood collection unit, or wherein the light emission unit is a separable component from the blood collection unit.

20. A kit comprising:
the device of claim 12; and
one or more of:
instructions for applying at least the blood collection unit to a skin surface and activating the light source to emit electromagnetic radiation to the skin surface;
an application for displaying the instructions or progress updates on a blood collection process in real-time, instructions for obtaining the application, or a link to obtain the application;
a disinfectant wipe;
a bandage;
a blood collection container;
a hardware device for sample monitoring during collection or shipping;
a centrifuge; or
shipping materials.
